(19) United States
(12) Patent Application Publication
(10) Pub. No.: US 2023/0380705 A1
(43) Pub. Date: Nov. 30, 2023

(54) CLOSED LOOP BLOOD PRESSURE CONTROL

(71) Applicant: Medtronic, Inc., Minneapolis, MN (US)

(72) Inventors: Richard J. O'Brien, Hugo, MN (US); Todd M. Zielinski, McGrath, MN (US); Nathan A. Torgerson, Andover, MN (US); Lilian Kornet, Eijsden (NL); Richard N. Cornelussen, Maastricht (NL); Shantanu Sarkar, Roseville, MN (US); Veronica Ramos, Miami, FL (US); Douglas A. Hettrick, Andover, MN (US); Yong K. Cho, Excelsior, MN (US)

(21) Appl. No.: 18/141,953

(22) Filed: May 1, 2023

Related U.S. Application Data
(60) Provisional application No. 63/347,495, filed on May 31, 2022.

(51) Int. Cl. A61B 5/021; A61B 5/0205; A61B 5/024; A61B 5/00

(52) U.S. Cl. CPC A61B 5/02125; A61B 5/0205; A61B 5/02438; A61B 5/4839

(57) ABSTRACT

A system may measure, by one or more sensors, a biometric parameter associated with a subject. The system may determine values of a control parameter based on measuring the biometric parameter. The control parameter may include blood pressure of the subject. The system may perform a control measure based on a comparison of the values of the control parameters to a threshold. Performing the control measure may include delivering therapy treatment to the subject or outputting a notification indicating an action associated with treating a medical condition. Measuring the biometric parameter, determining the values of the control parameter, and performing the control measure may be in response to one or more trigger criteria.

### CROSS-REFERENCE TO RELATED APPLICATIONS

The present application claims the benefit of U.S. Provisional Application Ser. No. 63/347,495 filed May 31, 2022. The entire disclosure of the application listed is hereby incorporated by reference, in its entirety, for all that the disclosure teaches and for all purposes.

### FIELD OF INVENTION

The present disclosure is generally directed to medical monitoring, and relates more particularly to blood pressure measurement and therapy control.

### BACKGROUND

Some systems may support medical monitoring of patients. In some cases, the systems may deliver therapy to patients in association with blood pressure control. Treatment techniques for providing closed loop blood pressure control are desired.

### BRIEF SUMMARY

Example Aspects of the Present Disclosure Include:

A system including: a processor; and a memory storing instructions thereon that, when executed by the processor, cause the processor to: measure, by one or more sensors, at least one biometric parameter associated with a subject; determine values of one or more control parameters based on measuring the at least one biometric parameter; and perform a control measure based on a comparison of the values of the one or more control parameters to a threshold.

Any of the aspects herein, wherein: determining the values of the one or more control parameters includes deriving the values of the one or more control parameters based on measuring the at least one biometric parameter; and the one or more control parameters include blood pressure of the subject.

Any of the aspects herein, wherein the instructions are further executable by the processor to: perform a second control measure based on a set of data points associated with the at least one biometric parameter.

Any of the aspects herein, wherein: the one or more sensors include an optical sensor, an ultrasound sensor, or both; and the at least one biometric parameter includes: a diameter associated with an anatomical element of the subject, a flow through the anatomical element, or both; or a pulse wave velocity value associated with the anatomical element.

Any of the aspects herein, wherein: the one or more sensors include an optical sensor; and the at least one biometric parameter includes a characteristic of a pulse associated with an anatomical element of the subject.

Any of the aspects herein, wherein: the one or more sensors include an impedance sensor; and the at least one biometric parameter includes a characteristic of a pulse of an impedance measurement associated with an anatomical element of the subject.

Any of the aspects herein, wherein: the one or more sensors include a subcutaneous blood pressure sensor; the at least one biometric parameter includes blood pressure associated with the subject; and the one or more control parameters include a characteristic associated with the blood pressure.

Any of the aspects herein, wherein the at least one biometric parameter includes: a pulse shape, a heart rate, blood volume, a flow rate, a pressure, or a combination thereof in association with an anatomical element of the subject. Any of the aspects herein, wherein the at least one biometric parameter includes: a first marker of a kidney function associated with blood pressure of the subject, a second marker of a thyroid function associated with the blood pressure, or a combination thereof.

Any of the aspects herein, wherein: the at least one biometric parameter includes an optical signal associated with an anatomical element, an electrical impedance associated with the anatomical element, or a pressure associated with the anatomical element; determining the values of the one or more control parameters includes deriving a waveform representation of the optical signal, the electrical impedance, or the pressure; and performing the control measure is based on comparing one or more characteristics of the waveform representation to a set of threshold criteria.

Any of the aspects herein, wherein performing the control measure includes: outputting a control signal to a device; and at least one of: delivering, by the device, therapy treatment to the subject based on the control signal; and outputting, by the device, a notification based on the control signal, wherein the notification includes an indication of one or more actions associated with treating a medical condition.

Any of the aspects herein, wherein performing the control measure includes: selecting a mode of a device configured for delivering therapy treatment to the subject, the device including a pacemaker, a neurostimulator, a drug pump, or a mechanical device; and delivering the therapy treatment to the subject based on the mode.

Any of the aspects herein, wherein the instructions are further executable by the processor to: set a mode associated with performing the control measure, wherein setting the mode is based on a value of the at least one biometric parameter, a value of the one or more control parameters, or both.

Any of the aspects herein, wherein measuring the at least one biometric parameter, determining the values of the one or more control parameters, and performing the control measure is in response to one or more trigger criteria.

Any of the aspects herein, wherein the one or more trigger criteria include at least one of: temporal criteria; a change in posture of the subject; a change in value of the at least one biometric parameter; and a change in activity level or activity status associated with the subject.

Any of the aspects herein, wherein the one or more trigger criteria include at least one of: a control signal associated with delivering therapy treatment to the subject; and delivery of the therapy treatment by a device.

Any of the aspects herein, wherein: the one or more trigger criteria include two or more trigger criteria; and the instructions are further executable by the processor to: derive a composite trigger value based on the two or more trigger criteria and respective weights of the two or more trigger criteria, wherein performing the control measure is in response to the composite parameter value satisfying one or more threshold values.

Any of the aspects herein, further including: providing a set of values of the at least one biometric parameter to a machine learning model, wherein determining the values of the one or more control parameters includes deriving, by the machine learning model, the values of the one or more control parameters in response to the machine learning model processing the set of values of the at least one biometric parameter.

Any of the aspects herein, wherein measuring the at least one biometric parameter, determining the values of the one or more control parameters, and performing the control measure are associated with a closed loop operating mode of the system.

A method including: measuring, by one or more sensors, at least one biometric parameter associated with a subject; determining values of one or more control parameters based on measuring the at least one biometric parameter; and performing a control measure based on a comparison of the values of the one or more control parameters to a threshold.

A system including: one or more sensors; a device configured for delivering therapy treatment; a processor; and a memory storing instructions thereon that, when executed by the processor, cause the processor to: measure, by the one or more sensors, at least one biometric parameter associated with a subject; determine values of one or more control parameters based on measuring the at least one biometric parameter; and perform a control measure via the device based on a comparison of the values of the one or more control parameters to a threshold.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Embodiments of the invention described herein can be adapted and implemented with a wide variety of non-invasive blood pressure monitoring techniques, including for example, U.S. Patent Publication No. US20210193311A1 and U.S. patent application Ser. No. 17/201,437, the contents of which are each incorporated herein by reference.

Numerous additional features and advantages of the present disclosure will become apparent to those skilled in the art upon consideration of the implementation descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, implementations, and configurations of the disclosure, as illustrated by the drawings referenced below.

FIGS. 1A and 1B illustrate examples of a system according to at least one implementation of the present disclosure.

FIG. 2 illustrates an example process flow that support aspects of the present disclosure.

FIG. 3 illustrates an example process flow that supports aspects of the present disclosure.

### DETAILED DESCRIPTION

It should be understood that various aspects disclosed herein may be combined in different combinations than the combinations specifically presented in the description and accompanying drawings. It should also be understood that, depending on the example or implementation, certain acts or events of any of the processes or methods described herein may be performed in a different sequence, and/or may be added, merged, or left out altogether (e.g., all described acts or events may not be necessary to carry out the disclosed techniques according to different implementations of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., ARM Cortex-M3 or Cortex-M33 processors; Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements and/or or hardware subroutines implemented by the circuits or logic elements.

Before any implementations of the disclosure are explained in detail, it is to be understood that the disclosure is not limited in its application to the details of construction and the arrangement of components set forth in the following description or illustrated in the drawings. The disclosure is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Blood pressure control is important for treating a subject for hypertension, heart failure, and kidney failure, and during and post-surgery. Some medical treatment approaches may include stimulation therapies (e.g., neuromodulatory therapies, deep brain stimulation (DBS) therapies, dorsal root ganglion (DRG) stimulation therapies, spinal cord stimulation (SCS) therapies, etc.) and drug pump therapies supportive of controlling blood pressure (e.g., increasing or decreasing blood pressure). In some cases, the therapies in such treatment approaches are open loop in that blood pressure is not continuously measured. For example, such therapies periodically measure blood pressure using a blood pressure cuff. Such non-continuous (e.g., periodic, open loop) measurements are unable to account for changes in blood pressure due to activity of a subject, posture changes of the subject, or other factors that can affect the biometric parameter of the patient.

In some cases, blood pressure may change as a consequence of stimulation therapies, drug therapies, or electrical stimulation. For example, for a drug delivery device (e.g., a implanted drug pump, a wearable pump, etc.) capable of delivering a drug that can affect blood pressure, aspects of the present disclosure support combining the drug delivery with spinal stimulation that is used to control blood pressure. Accordingly, for example, aspects of the present disclosure support modifying parameters associated with the spinal stimulation to avoid either hypotension or hypertension as a result of the drug bolus. Additionally, or alternatively, once a system learns the physiological response of a patient to a drug bolus, the system may titrate the drug delivery at a rate that mitigates or does not cause hypotension or hypertension. In some example implementations, the system may titrate the drug delivery without stimulating the spinal cord to change blood pressure.

Continuous blood pressure measurement techniques may be implemented using a pressure sensor within the blood pool. While technically challenging, example sensors capable of implementing such measurements include Abbott's CardioMEMS sensor, Remon ImPressure/RemonCHF from Remon Medical Technology (acquired by Boston Scientific, Natwick, MA, USA), CoraVie medical, Enter Vectorious, Medtronic's Chronicle devices. In contrast, passive pressure sensors utilized in combination with an external reader are of extremely limited utility in providing a control signal for blood pressure stimulation therapy. Although some implementations of continuous blood pressure measurement, as provided by active sensors (e.g., Medtronic's Chronicle devices), may be used for closed loop control, such implementations may be relatively invasive to a subject.

Aspects of the present disclosure support implementations of measuring ambulatory blood pressure using subcutaneous blood pressure sensors or wearable blood pressure sensors. Such implementations may acquire measurements near continuously. For example, such implementations may include automatically determining blood pressure (e.g., through directly measuring blood pressure, through deriving blood pressure based on biometric parameters of a subject, etc.) at temporal intervals that support closed loop blood pressure control therapy. For example, such implementations may include determining blood pressure according to a frequency (e.g., quantity of times during a temporal period) that supports closed loop blood pressure control therapy. Based on the blood pressure (measured and/or derived), the system may perform one or more control measures, aspects of which are later described herein. The term “subcutaneous” may refer to being situated, positioned, or applied at a location under the skin.

In an example implementation, a system of the present disclosure may include a subcutaneous sensor (or combination of subcutaneous sensors) placed near one or more anatomical elements (e.g., an artery, a vein, etc.) of a subject. For example, an example implementation may include a subcutaneous sensor located within a threshold distance of a vein and a subcutaneous sensor located within a threshold distance of an artery. Using measurements provided by the subcutaneous sensor(s), the system may derive a blood pressure value associated with the subject. For example, the system may derive blood pressure (e.g., relative or absolute) based on measurements provided by the subcutaneous sensor(s).

Accordingly, for example, the system may provide a complete picture of a cardiovascular system for closed loop control. For example, the system may support venous pressure control. The system may measure and evaluate preload pressure (venous) and afterload pressure (arterial). The term “preload” refers to the initial stretching of cardiac myocytes (muscle cells) prior to contraction and is related to ventricular filling. The term “afterload” refers to the force or load against which the heart has to contract to eject the blood.

Examples of subcutaneous sensors supported by the present disclosure are described herein. A subcutaneous sensor may be an optical sensor capable of measuring a diameter of the artery using optical techniques. For example, the subcutaneous sensor may emit light toward the artery, and the subcutaneous sensor may collects the reflected light from red blood cells included in blood vessels.

In another example, the subcutaneous sensor may be capable of emitting and receiving ultrasound waves, and the subcutaneous sensor may measure the diameter of an anatomical element (e.g., artery, vein) using ultrasound techniques. Further, for example, the subcutaneous sensor may use ultrasound techniques to measure flow (e.g., a flow rate) through the anatomical element (e.g., artery, vein).

In some aspects, the subcutaneous sensor may wirelessly communicate data to the system. For example, the subcutaneous sensor may transmit data (indicative of the diameter and the flow through the artery and/or vein) to the system, and the system may derive blood pressure based on the data. Additionally, or alternatively, the subcutaneous sensor may be capable of calculating the blood pressure of the artery based on the data, and the subcutaneous sensor may transmit data (indicative of the calculated blood pressure) to the system.

Based on the blood pressure, the system may perform one or more control measures. An example control measure includes delivering therapy to the subject via a device (e.g., a stimulation device, a drug delivery device, etc.). Another example control measure includes outputting a notification indicating one or more actions (e.g., take prescribed medication, perform a prescribed action, etc.) associated with treating a medical condition. In some examples, a prescribed action may include performing a breathing technique (e.g., deep breathing, breathing through an inspiratory resistive valve, etc.) prior to standing to increase preload and reduce the effects of orthostatic hypotension. Example aspects of the control measures are later described herein.

In another example implementation, the subcutaneous sensor may be an optical blood pressure sensor capable of detecting pulse waves associated with the artery. Based on a pulse shape associated with a detected pulse wave, the system may derive blood pressure (e.g., relative or absolute). In some aspects, the system may derive the blood pressure using a machine learning algorithm. Based on the blood pressure, the system may perform one or more control measures, aspects of which are later described herein.

In another example implementation, the subcutaneous sensor may be an impedance sensor. For example, the subcutaneous sensor may be a bio-impedance sensor capable of measuring an electrical impedance. Based on a pulse shape of an impedance measurement provided by the subcutaneous sensor, the system may derive blood pressure (e.g., relative or absolute). In some aspects, the system may derive the blood pressure using a machine learning algorithm. For example, the system may implement machine learning algorithms which identify and select patient-dependent fiducial points, based on which the machine learning algorithms may derive blood pressure. In an example, the system may convert the data values corresponding to the fiducial points to blood pressure.

The system may perform one or more control measures as described herein, based on the blood pressure (e.g., as derived by the system). Accordingly, for example, the system may utilize the derived blood pressure as a means of controlling a device in association with controlling blood pressure. The system may apply the derived blood pressure as a control signal.

Additionally, or alternatively, the system may perform one or more control measures based directly on the fiducial points identified and selected by the machine learning algorithms. For example, the system may refrain from converting the data values corresponding to the fiducial points to blood pressure. In an example, the system may apply the fiducials, or a subset of the fiducials, as a control signal for implementing a control measure. Accordingly, for example, the system may utilize the fiducial points directly as a means of control.

In another example implementation, the system may monitor activation of pressoreceptor regions (e.g., baroreceptors) of the subject. Baroreceptors include afferent nerves and sensory nerve endings that are sensitive to the stretching of the arterial walls (e.g., resulting from increased blood pressure from within). Baroreceptors function as the receptor of a central reflex mechanism that activates to reduce the pressure. In response to sensing the activation or firing of a baroreceptor, the system may perform one or more control measures described herein. In some aspects, the system may perform a control measure in response to the frequency and/or patterns associated with activation of the baroreceptors satisfying one or more target criteria. Accordingly, for example, the system may use sensed baroceptor firing (e.g., frequency, patterns) as a control signal. In some cases, the system may detect baroceptor firing to confirm whether a drug bolus from a drug pump has been delivered to the subject.

In some example implementations, the system may support continuous blood pressure measurement while the physical activity of a patient is below a threshold value (e.g., while the patient is not vigorously active). In some examples, the system may derive such continuous blood pressure measurements based on optical signals acquired via a sensor device (e.g., a wearable device, an implanted device, etc.). For example, the system may apply a machine learning algorithm capable of deriving blood pressure values from the shape of an optical signal throughout a cardiac cycle. Additionally, or alternatively, the system may derive blood pressure values based on the shape of an impedance waveform (e.g., as provided by a sensor device) throughout a cardiac cycle.

In some example implementations the system may divide impedance into a real (R) component and a reactive (Xc) component for analysis. For example, the system may associate the real (R) component of impedance with blood volume and associate the reactive (Xc) component of impedance with change in the vessel wall. In some aspects, the system may provide the real (R) and reactive (Xc) components into an AI algorithm, which may support improved sensitivity and specificity in association with deriving blood pressure values.

Aspects of the present disclosure support combining continuous blood pressure measurement (or blood pressure derivation) techniques with stimulation therapies and/or other therapies that affect (e.g., reduce) blood pressure, thereby providing closed loop control of blood pressure.

Examples of the sensor device described herein include an optical or impedance sensor integrated with (e.g., contained within) a therapy device. The term “therapy device” may be used interchangeable with the term “stimulation device”. In some alternative and/or additional aspects, the sensor device may be physically separate from the therapy device. In an example implementation, the system may include a blood pressure sensor located near or at an artery that is of sufficient size for acquiring blood pressure measurements, and the system may include a therapy device at a location different from that of the blood pressure sensor. That is, for example, the blood pressure sensor may be located near or at an artery that is not the best implant location for the therapy device (e.g., based on characteristics of the artery, vein, etc.). The blood pressure sensor (either as a wearable or an implant) may be placed at a location for sensing, and the blood pressure sensor may communicate (e.g., via a wireless or wired connection) with the therapy device to provide a control signal.

Implementations of the present disclosure provide technical solutions to one or more of the problems of blood pressure control. For example, aspects of the closed loop blood pressure control system may provide continuous or near-continuous measurements capable of accounting for changes in blood pressure due to activity of a subject, posture changes of the subject, or other factors that can affect biometric parameters of a subject. The techniques described herein provide technical solutions to controlling blood pressure as described herein.

Aspects of the present disclosure support closed loop blood pressure control. For example, aspects of the present disclosure support monitoring of biometric parameters and providing therapeutic treatments and/or patient notifications supportive closed loop control of blood pressure. Example aspects of a system (e.g., sensor devices, therapeutic devices, and stimulation therapies supported thereby) providing closed loop control of blood pressure are described herein with respect to the following figures.

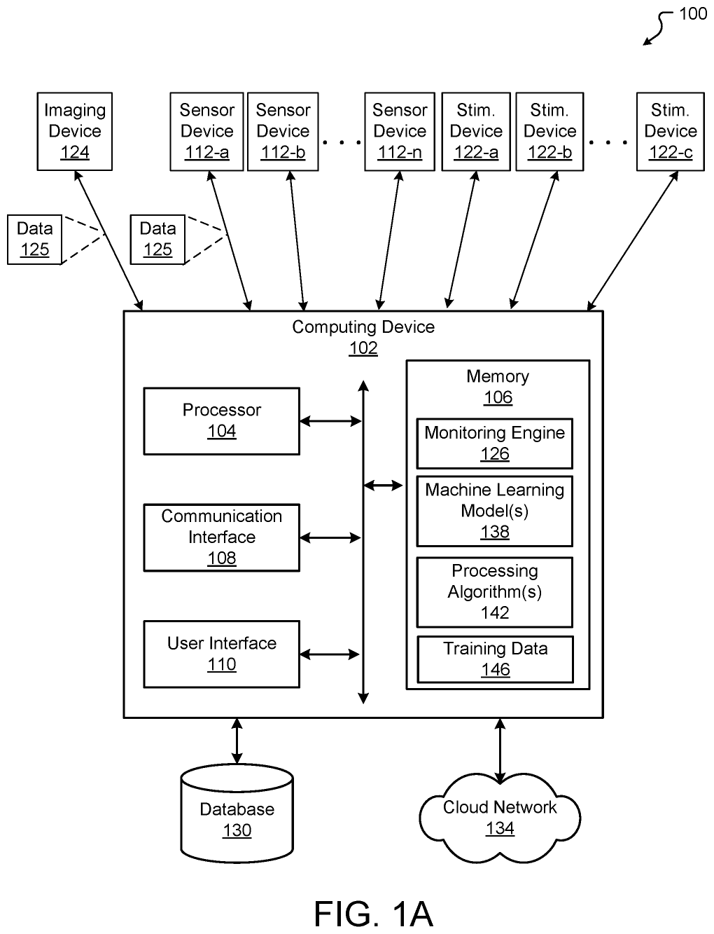

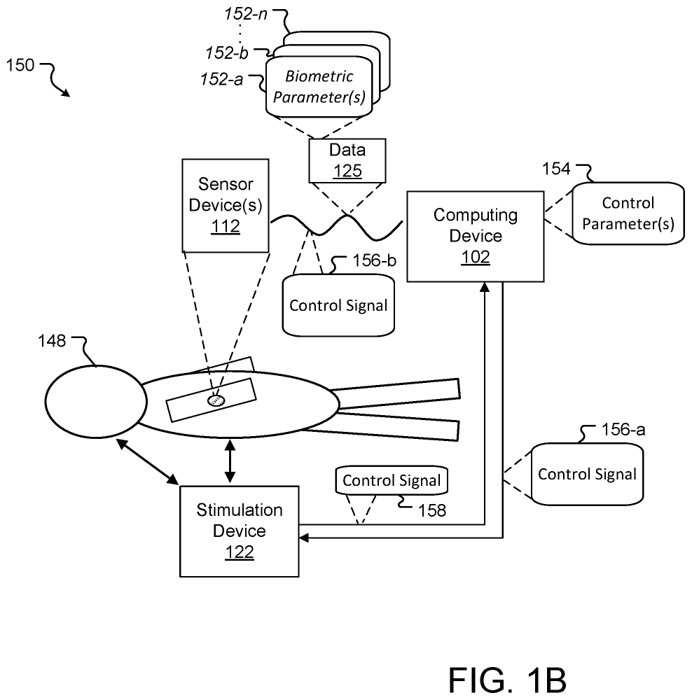

FIG. 1A illustrates an example of a system 100 that supports aspects of the present disclosure. The system 100 may be a closed loop blood pressure control system supportive of aspects of the present disclosure.

The system 100 includes a computing device 102, sensor devices 112 (e.g., sensor device 112-a through sensor device 112-n, where n is an integer value), a stimulation device 122 (or multiple stimulation devices 122, for example, stimulation device 122-a through stimulation device 122-n), imaging device 124, a database 130, a cloud network 134 (or other network), and/or a server (not illustrated). Systems according to other implementations of the present disclosure may include more or fewer components than the system 100. For example, the system 100 may omit and/or include additional instances of the sensor devices 112, stimulation device 122, imaging device 124, one or more components of the computing device 102, the database 130, the cloud network 134, and/or the server. In an example, the system 100 may omit any instance of the sensor devices 112, stimulation device 122, imaging device 124, one or more components of the computing device 102, the database 130, and the cloud network 134. The system 100 may support the implementation of one or more other aspects of one or more of the methods disclosed herein.

The computing device 102 includes a processor 104, a memory 106, a communication interface 108, and a user interface 110. Computing devices according to other implementations of the present disclosure may include more or fewer components than the computing device 102. The computing device 102 may include, for example, electronic circuitry and software supportive of closed loop blood pressure control. The computing device 102 may support generating and outputting control signals, outputting notifications (e.g., via a user interface 110) and/or delivering therapy treatment (e.g., via the stimulation device 122) tailored for providing therapy and/or alerts specific to the subject.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from components (e.g., sensor device 112, stimulation device 122, imaging device 124, database 130, cloud network 134, etc.) of the system 100.

The memory 106 may be or include RAM, DRAM, SDRAM, other solid-state memory, any memory described herein, or any other tangible, non-transitory memory for storing computer-readable data and/or instructions. The memory 106 may store information or data associated with completing, for example, any step of the method 400 described herein, or of any other methods. The memory 106 may store, for example, instructions and/or machine learning models (e.g., machine learning model(s) 138) that support one or more functions of the sensor device 112, stimulation device 122, imaging device 124, and/or the computing device 102. For instance, the memory 106 may store content (e.g., instructions and/or machine learning model(s) 138) that, when executed by the processor 104, enable monitoring of biometric parameters, deriving and monitoring of blood pressure, data analysis, and notification generation (e.g., by a monitoring engine 126). Such content, if provided as in instruction, may, in some implementations, be organized into one or more applications, modules, packages, layers, or engines.

Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the sensor device 112, imaging device 124, stimulation device 122, the database 130, and/or the cloud network 134.

The computing device 102 may also include a communication interface 108. The communication interface 108 may be used for receiving data or other information from an external source (e.g., sensor device 112, stimulation device 122, imaging device 124, database 130, cloud network 134, and/or any other system or component separate from the system 100), and/or for transmitting instructions, data (e.g., biometric parameter measurements, blood pressure measurements, etc.), or other information to an external system or device (e.g., another computing device 102, sensor device 112, stimulation device 122, imaging device 124, database 130, the cloud network 134, and/or any other system or component not part of the system 100). The communication interface 108 may include one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, ultrasound communications, and tissue conductance communication (TCC) or intra-body communication (IBC) using tissue in the human body as a medium for electrical signals, and so forth). In some implementations, the communication interface 108 may support communication between the device 102 and one or more other processors 104 or computing devices 102, whether to reduce the time needed to accomplish a computing-intensive task or for any other reason.

The computing device 102 may also include one or more user interfaces 110. The user interface 110 may be or include a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some implementations, the user interface 110 may support user modification (e.g., by a surgeon, medical personnel, a patient, etc.) of instructions to be executed by the processor 104 according to one or more implementations of the present disclosure, and/or to user modification or adjustment of a setting of other information displayed on the user interface 110 or corresponding thereto.

In some implementations, the computing device 102 may utilize a user interface 110 that is housed separately from one or more remaining components of the computing device 102. In some implementations, the user interface 110 may be located proximate one or more other components of the computing device 102, while in other implementations, the user interface 110 may be located remotely from one or more other components of the computer device 102.

The sensor device 112 may be operable to measure biometric parameters associated with a subject. Non-limiting examples of the one or more biometric parameters measurable by the sensor device 112 include a pulse shape, a heart rate, blood volume, a flow rate, a pressure, or a combination thereof in association with an anatomical element of the subject, examples of which are described herein. Other non-limiting examples of the one or more biometric parameters include a first marker of a kidney function associated with blood pressure (e.g., high blood pressure, measured blood pressure that exceeds a threshold value) of the subject, a second marker of a thyroid function associated with the blood pressure, or a combination thereof.

Examples of the sensor device 112 include an optical sensor, an impedance sensor, a subcutaneous sensor blood pressure sensor, an ultrasound sensor, a differential pulse transit time sensor, or the like, and are not limited thereto.

In some cases, the sensor device 112 may image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an sensor device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some implementations, a first sensor device 112 may be used to obtain first image data (e.g., a first image) at a first time, and a second sensor device 112 may be used to obtain second image data (e.g., a second image) at a second time after the first time. The sensor device 112 may be capable of taking a 2D image or a 3D image to yield the image data. The sensor device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other sensor device 112 suitable for obtaining images of an anatomical feature of a patient. The sensor device 112 may be contained entirely within a single housing, or may comprise a transmitter/emitter and a receiver/detector that are in separate housings or are otherwise physically separated.

The sensor device 112 may support determining blood pressure from a photoplethysmographic (PPG) signal, which may provide for a non-invasive method for measuring blood pressure continuously. Aspects of the present disclosure may include using continuous non-invasive blood pressure (CNIBP) measurement as an alternative to invasive blood pressure measurement techniques. In some aspects, the system 100 may support CNIBP using an AI model where the inputs include pulse wave velocity as determined at two locations (e.g., two distinct locations at an artery, vein, etc.).

The computing device 102 may measure or derive blood pressure using techniques described herein, based on data (e.g., measured biometric parameters) provide by one or more sensor devices 112.

The imaging device 124 may be, for example, a depth camera supportive of object sensing and/or depth sensing. For example, the imaging device 124 may facilitate recognition, registration, localization, mapping, and/or tracking of a subject (e.g., patient anatomical features, patient movement, patient posture, etc.). For example, the imaging device 124 may identify points in space corresponding to an object (e.g., a patient, an anatomical element of the patient, etc.) based on the distance the point is from the imaging device 124 (e.g., a depth differential between the object and the imaging device 124). The imaging device 124 may support locating objects based on depth, for example, in real-time or near real-time.

The imaging device 124 (e.g., with or without the computing device 102) may process captured data (e.g., depth data) in real-time or near real-time and provide motion data based on data captured at different temporal instances. Accordingly, for example, the imaging device 124 may support motion sensing. For example, the imaging device 124 may track the movement of a subject and/or other objects in the field of view of the imaging device 124. In some aspects, the imaging device 124 may support registry and/or tracking of an object relative to a topography of the subject. The imaging device 124 may provide data 125 to the computing device 102. The data 125 may include depth data and/or motion data described herein. In some aspects, the imaging device 124 may be integrated with or separate from the sensor device 112.

The stimulation devices 122 may be external or internal to the subject. In some examples, the stimulation devices 122 may include aspects (e.g., components, functionalities, etc.) of the computing device 102. In some cases, the stimulation devices 122 may be a cardiac pacemaker, a cardioverter-defibrillator, a drug delivery device, a biologic therapy device, a monitoring or therapeutic device, or the like. The system 100 may include any combination of stimulation devices 122 described herein.

In an example implementation, the stimulation device 122-a may be a neurostimulation device (e.g., a neurostimulator) including stimulation circuitry, sensing circuitry, and a stimulation controller (not illustrated). In some examples, the stimulation device 122-a may be an implanted neurostimulator (e.g., an implanted pulse generator (IPG))).

In another example implementation, the stimulation device 122-b may be an external pressure garment. The computing device 102 may automatically change the amount of pressure that the stimulation device 122-b provides, based on measured or derived blood pressure values.

In another example implementation, in an analogous fashion to an insulin pump using blood glucose measurements to titrate insulin delivery, the stimulation device 122-c may be a drug pump. For example, the stimulation device 122-c may be a wearable or implantable pump capable of titrating the delivery of drugs to change blood pressure for hypertension or heart failure. As an example, many therapeutic drugs have as potential side effects changes in blood pressure. The system 100 may incorporate a sensor device 112 (e.g., a wearable or implanted pressure sensor) to provide warnings of undesired changes in blood pressure and, in some cases, halt or slow the administration of the drug being delivered by the stimulation device 122-c

In another example implementation, the stimulation device 122-d may be a pacemaker capable of providing electrical impulses to an anatomical element (e.g., ventricle(s) of the heart) of a subject in response to control signals provided by the computing device 102. Cardiac pacing is an intervention that helps return the heartbeat return to a target pace when the heartbeat has been temporarily out of rhythm. Cardiac pacing rates are based on surrogate measure of metabolic needs such as activity. The computing device 102 may monitor blood pressure (e.g., measure or derive blood pressure as described herein) and, using a rate response algorithm and the stimulation device 122-d, adjust cardiac pacing rates to meet metabolic needs while maintaining blood pressure within a normal blood pressure range (e.g., systolic pressure of less than 120 and a diastolic pressure of less than 80). In some aspects, the computing device 102 may adjust cardiac pacing rates to meet metabolic needs while controlling blood pressure so as to avoid dangerous ranges (e.g., low blood pressure, high blood pressure, etc.). In some cases, the computing device 102 may monitor (e.g., measure or derive) blood pressure and/or adjust cardiac pacing rates based on additional factors such as the posture (e.g., sitting, standing, lying down, etc.) of a subject.

In some example implementations, the system 100 may be a drug pump and stimulator system including both the stimulation device 122-c (e.g., drug pump) and the stimulation device 122-d (e.g., pacemaker). Many therapeutic drugs have as potential side effects changes in blood pressure. In cases where it is undesirable to discontinue delivery of a drug to a subject (e.g., due to a negative health outcome) via the stimulation device 122-c, the computing device 102 may apply one or more stimulation therapies described herein to counter changes in blood pressure resulting from the drug delivery. For example, using the stimulation device 122-d, the computing device 102 may bring the patient's systemic blood pressure to target levels.

Example stimulation therapies capable of controlling blood pressure and that may be incorporated in the present disclosure include inducing short atrioventricular (AV) delays to decrease systolic volume and thereby decreasing cardiac output (CO) to decrease mean arterial pressure (MAP), where MAP is the product of CO and total peripheral vascular resistance (TPR).

Examples of other therapies capable of controlling blood pressure and that may be incorporated in the present disclosure include titrating stimulation parameters for dorsal root ganglion (DRG) stimulation or lateral epidural stimulation. For example, the system 100 may support blood pressure control using a stimulation device 122 that targets afferent sensory fibers kidneys in the dorsal root at the T12 vertebra of the body. In some cases, the system 100 may support DRG stimulation at the T7 through T12 vertebrae to lower blood pressure via the liver. In some other cases, the system 100 may support targeting T5 through T9 vertebrae to affect sensory fibers of greater splanchnic nerve affecting the splanchnic capacitance veins (which contain about 25% of the total blood volume). The reservoir associated with the splanchnic capacitance veins is the source of additional blood volume during exercise or stress.

Examples of other therapies capable of controlling blood pressure and that may be incorporated in the present disclosure include vagal nerve stimulation. Examples of other therapies capable of controlling blood pressure and that may be incorporated in the present disclosure include diaphragm stimulation. For example, the system 100 may support stimulating the diaphragm to change intrathoracic pressure and increase venous return, which will increase blood pressure. Accordingly, for example, the system 100 may use a sensor device 112 (e.g., subcutaneous blood pressure sensor) as a control measure for monitoring intrathoracic pressure.

Examples of other therapies capable of controlling blood pressure and that may be incorporated in the present disclosure include baroreceptor stimulation. Baroreceptors are located at the carotid and aortic bifurcation. The system 100 may support stimulating, using one or more stimulation devices 122, receptors in the neck of a subject (i.e. carotid bifurcation) in association with hypertension treatment.

Examples of other therapies capable of controlling blood pressure and that may be incorporated in the present disclosure include acute blood shunting from organs or muscular groups on demand for changes in circulation demand (e.g., in association with moving from a sitting position to a standing position). The system 100 may support taking blood from the venous reservoir by stimulating, using one or more stimulation devices 122, the nerve feeding the muscle group.

Examples of other therapies capable of controlling blood pressure and that may be incorporated in the present disclosure include increasing stroke volume. For example, the system 100 may support stimulating, using one or more stimulation devices 122, blood volume reservoirs (e.g., spleen, liver, central venous vascular, viscera, etc.) to introduce more blood volume into circulation, thereby increasing stroke volume.

The system 100 may include any combination of sensor devices 112, stimulation devices 122, and stimulation therapies described herein.

The monitoring engine 126 may determine values of one or more control parameters based the data 125 (e.g., biometric parameters, motion data, etc.) associated with a subject. For example, the data 125 may values of biometric parameters (as measured by the sensor devices 112) and motion data (as provided by the imaging device 124).

The monitoring engine 126 may perform a control measure based on a comparison of the values of the one or more control parameters to a threshold. For example, the monitoring engine 126 may transmit a control signal to a stimulation device 122 for delivering a treatment to the subject. In another example of a control measure, the monitoring engine 126 may generate and output a notification via user interface 110. In some aspects, the monitoring engine 126 may transmit a control signal a sensor device 112 for measuring a biometric parameter.

The processor 104 may utilize data stored in memory 106 as a neural network. The neural network may include a machine learning architecture. In some aspects, the neural network may be or include one or more classifiers. In some other aspects, the neural network may be or include any machine learning network such as, for example, a deep learning network, a convolutional neural network, a reconstructive neural network, a generative adversarial neural network, or any other neural network capable of accomplishing functions of the computing device 102 described herein. Some elements stored in memory 106 may be described as or referred to as instructions or instruction sets, and some functions of the computing device 102 may be implemented using machine learning techniques.

For example, the processor 104 may support machine learning model(s) 138 which may be trained and/or updated based on data (e.g., training data 146) provided or accessed by any of the computing device 102, the sensor device 112, the stimulation device 122, the imaging device 124, the database 130, and/or the cloud network 134. The machine learning model(s) 138 may be built and updated by the monitoring engine 126 based on the training data 146 (also referred to herein as training data and feedback).

For example, the machine learning model(s) 138 may be trained with one or more training sets included in the training data 146. In some aspects, the training data 146 may include multiple training sets. In an example, the training data 146 may include a first training set that includes biometric data (e.g., pulse shape, heart rate, blood volume, flow rate, pressure, a marker of kidney function, a marker of thyroid function) associated with one or more medical conditions (e.g., high blood pressure, low blood pressure, etc.) described herein. In some aspects, the biometric data included in the first training set may be associated with confirmed instances (e.g., by a healthcare provider, a patient, etc.) of the one or more medical conditions.

The training data 146 may include a second training set that includes control parameter data (e.g., blood pressure values) associated with one or more medical conditions described herein. In some aspects, the control parameter data included in the second training set may be associated with confirmed instances (e.g., by a healthcare provider, a patient, etc.) of the one or more medical conditions.

The training data 146 may include a third training set that includes positional data (e.g., posture), and/or activity data (e.g., physical activity, motion data, etc.) of a subject in association with one or more medical conditions described herein. In some aspects, the positional data and/or activity data included in the third training set may be associated with confirmed instances (e.g., by a healthcare provider, a patient, etc.) of the one or more medical conditions.

In some examples, based on the data included in the training sets, the neural network may generate one or more algorithms (e.g., processing algorithms 142) supportive of closed loop blood pressure control described herein.

The database 130 may store, for example, one or more surgical plans; one or more treatment plans; one or more images useful in connection with a surgery to be completed by or with the assistance of one or more other components of the system 100; and/or any other useful information.

The database 130 may additionally or alternatively store, for example, location or coordinates of devices (e.g., sensor devices 112, imaging device 124, stimulation devices 122, etc.) of the system 100. The database 130 may be configured to provide any such information to the computing device 102 or to any other device of the system 100 or external to the system 100, whether directly or via the cloud network 134. In some implementations, the database 130 may include treatment information (e.g., for managing a medical condition, for example, hypertension, heart failure, kidney failure, etc.) and or historical physiological data (e.g., biometric parameters, blood pressure, etc.) associated with a patient. In some implementations, the database 130 may be or comprise part of a hospital image storage system, such as a picture archiving and communication system (PACS), a health information system (HIS), and/or another system for collecting, storing, managing, and/or transmitting electronic medical records of a patient.

In some aspects, the computing device 102 may communicate with a server(s) and/or a database (e.g., database 130) directly or indirectly over a communications network (e.g., the cloud network 134). The communications network may include any type of known communication medium or collection of communication media and may use any type of protocols to transport data between endpoints. The communications network may include wired communications technologies, wireless communications technologies, or any combination thereof.

Non-limiting examples of the computing device 102 may include, for example, personal computing devices or mobile computing devices (e.g., laptop computers, mobile phones, smart phones, smart devices, wearable devices, tablets, etc.). In some examples, computing device 102 may be operable by or carried by a human user. In some aspects, the computing device 102 may perform one or more operations autonomously or in combination with an input by the user.

Wired communications technologies may include, for example, Ethernet-based wired local area network (LAN) connections using physical transmission mediums (e.g., coaxial cable, copper cable/wire, fiber-optic cable, etc.). Wireless communications technologies may include, for example, cellular or cellular data connections and protocols (e.g., digital cellular, personal communications service (PCS), cellular digital packet data (CDPD), general packet radio service (GPRS), enhanced data rates for global system for mobile communications (GSM) evolution (EDGE), code division multiple access (CDMA), single-carrier radio transmission technology (1×RTT), evolution-data optimized (EVDO), high speed packet access (HSPA), universal mobile telecommunications service (UMTS), 3G, long term evolution (LTE), 4G, and/or 5G, etc.), Bluetooth®, Bluetooth® low energy, Wi-Fi, radio, satellite, infrared connections, and/or ZigBee® communication protocols.

The Internet is an example of the communications network that constitutes an Internet Protocol (IP) network consisting of multiple computers, computing networks, and other communication devices located in multiple locations, and components in the communications network (e.g., computers, computing networks, communication devices) may be connected through one or more telephone systems and other means. Other examples of the communications network may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communications network 120 may include of any combination of networks or network types. In some aspects, the communications network may include any combination of communication mediums such as coaxial cable, copper cable/wire, fiber-optic cable, or antennas for communicating data (e.g., transmitting/receiving data).

The computing device 102 may be connected to the cloud network 134 via the communication interface 108, using a wired connection, a wireless connection, or both. In some implementations, the computing device 102 may communicate with the database 130 and/or an external device (e.g., a computing device) via the cloud network 134.

The system 100 or similar systems may be used, for example, to carry out one or more aspects of any of the process flow 200 and process flow 300 described herein. The system 100 or similar systems may also be used for other purposes.

FIG. 1B illustrates an example 150 of the system 100 that supports providing closed loop blood pressure control of a subject 148 in accordance with aspects of the present disclosure.

A sensor device 112 may measure one or more biometric parameters 152 (e.g., biometric parameter 152-a through biometric parameter 152-n, where n is an integer value) associated with a subject 148. The sensor device 112 may include any combination of sensor devices 112 described herein. Non-limiting examples of the one or more biometric parameters 152 measured by the sensor devices 112 include a pulse shape, a heart rate, blood volume, a flow rate, a pressure, or a combination thereof in association with an anatomical element of the subject 148, examples of which are described herein. In an example, the sensor device 112 may provide data 125 (e.g., values of the biometric parameters 152, characteristics of the biometric parameters 152, posture, activity data, etc.) associated with a subject 148 to the computing device 102.

Based on the data 125, the computing device 102 may generate values associated with control parameters 154. In some cases, the computing device 102 may provide the data 125 (e.g., values of the biometric parameters 152, characteristics of the biometric parameters 152, etc.) to machine learning model 138. The computing device 102 may receive an output from the machine learning model 138 in response to the machine learning model 138 processing the data 125 (or a portion thereof). The output may include one or more values of the control parameters 154. Accordingly, for example, the machine learning model 138 may derive the values of the one or more control parameters 154 in response to the machine learning model 138 processing the values of the biometric parameters 152.

For example, the machine learning model 138 may implement machine learning algorithms which identify and select patient-dependent fiducial points from the data 125. Using the machine learning algorithms and the fiducial points, the machine learning model 138 may derive values of a control parameter 154 (e.g., blood pressure). In an example, the machine learning model 138 may convert the data values corresponding to the fiducial points to blood pressure.

The computing device 102 may perform a control measure based on a comparison of the one or more values of the control parameters 154 to a threshold. Examples of the biometric parameters 152, control parameters 154, and control measures are described herein. Aspects of the present disclosure support measuring the one or more biometric parameters 152, determining the values of the one or more control parameters 154, and/or performing the control measure in response to any combination of trigger criteria described herein.

In an example, the sensor device 112 may measure a biometric parameter 152 based on temporal criteria (e.g., expiry of a temporal value, for example, 15 minutes).

In some other aspects, the sensor device 112 may measure the biometric parameter 152 in response to detecting a change in posture of the subject 148 (e.g., from a sitting position to a standing position, from a position lying down to a sitting position, etc.). In an example, another sensor device 112 (e.g., an accelerometer, a gyroscope, etc.) or an imaging device 124 electrically coupled to the computing device 102 or in wireless communication with the computing device 102 may detect whether the changes in posture occur.

In some example aspects, the sensor device 112 may measure the biometric parameter 152 (e.g., biometric parameter 152-a, for example, pulse characteristic, electrical impedance, flow rate, pressure, etc.) in response to a change in value of another biometric parameter 152 (e.g., biometric parameter 152-b, for example, heart rate). In an example, another sensor device 112 (not illustrated) may detect the change in value of the other biometric parameter 152.

In some aspects, the sensor device 112 may measure the biometric parameter 152 in response to a change in a change in activity level (e.g., from a relatively low activity level to a relatively high activity level) associated with the subject 148. In some cases, the sensor device 112 may measure the biometric parameter 152 in response to a change in activity status (e.g., asleep, awake, at rest, exercising, etc.) of the subject 148.

In some aspects, trigger criteria based on which the sensor device 112 measures a biometric parameter 152 may include a control signal associated with performing a control measure. For example, the computing device 102 may perform a control measure that includes delivering treatment to the subject 148 via a stimulation device 122. In another example of a control measure, the computing device 102 may output a notification (e.g., via user interface 110 described with reference to FIG. 1A). Additionally, or alternatively, the computing device 102 may output a control signal to another computing device 102 (not illustrated), and the other computing device 102 may output the notification via a corresponding user interface. The notification may include any combination of visual, audible, and/or haptic alerts. In some aspects, the notification may include an indication of one or more actions (e.g., take prescribed medication, perform a prescribed action (for example, “walk for 10 minutes”), etc.) associated with treating a medical condition.

The stimulation device 122 may be any example stimulation device described herein (e.g., a pacemaker, a neurostimulator, a drug pump, a mechanical device, etc.). The computing device 102 may transmit a control signal 156-a to the stimulation device 122 for delivering the treatment to the subject 148. In response to the control signal 156-a, the stimulation device 122 may transmit a control signal 158 (to the computing device 102) to indicate that the stimulation device 122 is delivering/has delivered treatment to the subject 148.

The computing device 102 may transmit a control signal 156-b to the sensor device 112 for measuring the biometric parameter 152. In some cases, the computing device 102 may transmit the control signal 156-b to the sensor device 112 in combination with transmitting the control signal 156-a to the stimulation device 122. In some other aspects, the computing device 102 may transmit the control signal 156-b to the sensor device 112 in response to receiving the control signal 158 from the stimulation device 122.

Additionally, or alternatively, the computing device 102 may detect a therapeutic event, for example, delivery of the therapy treatment (e.g., using another sensor device 112 (not illustrated)). The computing device 102 may transmit the control signal 156-b in response to the detected therapeutic event. For example, the computing device 102 may transmit the control signal 156-b in response to detecting delivery of a therapy treatment (e.g., a drug, pump bolus, etc.) to the subject 148. The term “bolus” refers to the dose of a drug (e.g., insulin) that is specifically taken at meal times to help control blood glucose levels following a meal. The term “bolus” may refer to any relatively large drug delivery (as opposed to continuous delivery of relatively smaller amounts) and is not limited to insulin. Accordingly, for example, aspects of the present disclosure support utilizing therapeutic events as a trigger for activating one or more sensor devices 112 (and measuring biometric parameters 152), which may potentially lead to an alteration of therapy associated with the therapeutic events or lead to some other intervention. Aspects of the present disclosure support repetitive bolus delivery that can be administered upon a trigger. Some example implementations support basal-delivery and bolus upon a trigger.

Aspects of the present disclosure support multiple trigger criteria. In an example, the computing device 102 (or a server, not illustrated) may derive a composite trigger value based on a combination of trigger criteria described herein. For example, the computing device 102 may derive the composite trigger value based on multiple trigger criteria and respective weights thereof. In some aspects, the computing device 102 may performing a control measure in response to the composite parameter value satisfying a threshold value. In an example, increased current intensity associated with electrical stimulation may have a negative overall impact, as such an increase in intensity may overstimulate and unintentionally stimulate motor fibers, thereby counteracting sensory fiber stimulation. In an example implementation, aspects of the present disclosure support setting a target composite trigger value, using an equation to determine resulting output values based on multiple weighted factors, and comparing the resulting output values to the target composite trigger value.

In an example implementation, the sensor device 112 may include an optical sensor, an ultrasound sensor, or both. In an example, the biometric parameter 152 measured by the sensor device 112 may include a diameter associated with an anatomical element (e.g., an artery, vein, etc.) of the subject 148, a flow (e.g., a flow rate) through the anatomical element, or both. In another example, the biometric parameter 152 may include a pulse wave velocity value associated with the anatomical element. For example, in measuring the biometric parameter 152, sensor devices 112 respectively located at two locations at an artery may measure pulse wave velocity between the two locations.

In another example implementation, the sensor device 112 may include an optical sensor. In an example, the biometric parameter 152 measured by the sensor device 112 may include a pulse shape (e.g., generated based on measurements by the sensor device 112) associated with an anatomical element (e.g., an artery, vein, etc.) of the subject 148.

In some example implementations, the sensor device 112 may include an impedance sensor capable of providing electrical impedance measurements. In an example, the biometric parameter 152 measured by the sensor device 112 may include a pulse shape of an impedance measurement (e.g., provided by the impedance sensor) associated with an anatomical element of the subject 148.

In another example implementation, the sensor device 112 may include a subcutaneous blood pressure sensor. The biometric parameter 152 measured by the sensor device 112 may include blood pressure associated with the subject 148. In an example, the sensor device 112 may be located in the blood pool and capable of measuring blood pressure in association with the blood pool. Additionally, or alternatively, the sensor device 112 may be positioned against (e.g., pressed against) the wall of a blood vessel and capable of measuring a transmural pressure. The term “transmural pressure” may refer to pressure measured through the wall of the blood vessel.

In other example implementations, the biometric parameter 152 measured by the sensor device 112 may include a first marker of a kidney function associated with blood pressure (e.g., high blood pressure, measured blood pressure that exceeds a threshold value) of the subject, a second marker of a thyroid function associated with the blood pressure, or a combination thereof.

The computing device 102 may select a mode of the stimulation device 122 in association with delivering therapy treatment to the subject 148. For example, the stimulation device 122 may be a pacemaker, a neurostimulator, a drug pump, or a mechanical device, but is not limited thereto. The selected mode may include one or more settings (e.g., cardiac pacing rate, drug dosage, treatment intensity or frequency, etc.) with delivering therapy treatment via the stimulation device 122.

In another example, the computing device 102 may set different modes of therapy based on values of the biometric parameter(s) 152 and/or values of the control parameter(s) 154. For example, the computing device 102 may implement a first mode of therapy in association with treating high blood pressure. In some cases, the computing device 102 may implement a second mode of therapy in association with treating high heart rate and high blood pressure. In another example, the computing device 102 may implement a third mode of therapy in association with treating low heart rate and low blood pressure. In another example, the computing device 102 may implement a fourth mode of therapy in association with treating a high heart rate and low blood pressure. Each mode may include a corresponding set of parameters (e.g., parameters of a sensor device 112, parameters of a stimulation device 122, etc.). Aspects of the present disclosure support implementing any order or combination of the modes, for example, in association with prioritizing any of high blood pressure, low blood pressure, high heart rate, low heart rate, or combinations thereof.

In another example, the computing device 102 may select a mode associated with outputting notifications to the subject 148. The mode may include settings associated with delivery type (e.g., visual, audible, haptic, etc.), notification content, delivery frequency, or the like.

Other non-limiting examples of control measures that may be implemented by the computing device 102 include controlling pulse width of an electrical current administered to the subject 148, enabling a continuous mode or burst mode (e.g., bursts of pulses or signals), on-off cycling of the burst, intensity of an electrical current administered to the subject 148, selection of an electrode associated with administering treatment (e.g., electrical stimulation) to the subject 148, selection of a lead associated with administering the treatment, selection of a location (e.g., anatomical element, position on the subject 148, etc.) associated with administering the treatment, selection of a cathode-anode associated with administering the treatment, and selecting a pulse form of an electrical signal associated with administering the treatment.

In another example implementation, the computing device 102 may derive a waveform representation of a biometric parameter 152 (e.g., an optical signal associated with an anatomical element, an electrical impedance associated with the anatomical element, or a pressure associated with the anatomical element). The computing device 102 may compare one or more characteristics of the waveform representation to a set of threshold criteria (e.g., a target pulse shape, a target pattern, etc.). The computing device 102 may perform a control measure described herein (or refrain from performing a control measure) in response to a result of the comparison.

In some cases, the computing device 102 may derive a waveform representation of a biometric parameter 152 based on a diameter associated with the anatomical element and/or a flow rate through the anatomical element. For example, the computing device 102 may use the diameter to calculate arterial compliance (e.g., arterial elasticity and extensibility during a cardiac cycle). Together with average flow, the computing device 102 may use the arterial compliance in a Windkessel model to estimate local pressure associated with the anatomical element. In some aspects, the computing device 102 may use the flow rate to estimate average flow and wall shear rate.

Additionally, or alternatively, the computing device 102 may perform a control measure based on fiducial points (or a subset of the fiducial points) associated with the at least one biometric parameter 152. For example, the computing device 102 may refrain from converting the data values corresponding to the fiducial points to blood pressure. In an example, the computing device 102 may apply the fiducials (or subset of the fiducials) as a control signal. The computing device 102 may compare the fiducials to threshold criteria. Based on a result of the comparison, the computing device 102 may perform a control measure or refrain from performing a control measure.

Aspects of the present disclosure support surrogate physiology measurements associated with change in blood pressure. For example, a sensor device 112 may measure a biometric parameter 152-c (e.g., fluid status) and/or biometric parameter 152-d (e.g., S1 and S2 heart sounds). For a control parameter 154 (e.g., blood pressure) derived by the computing device 102 using biometric parameter 152-a (e.g., pulse characteristic, electrical impedance, flow rate, pressure, etc.), the computing device 102 may additionally derive the control parameter 154 using biometric parameter 152-c and/or biometric parameter 152-d. The computing device 102 may validate the control parameter 154 derived using biometric parameter 152-a, based on a comparison against the control parameter 154 as derived using biometric parameter 152-c and/or biometric parameter 152-d. Accordingly, for example, aspects of the system 100 support using the surrogate physiology in association with validating blood pressure measurements.

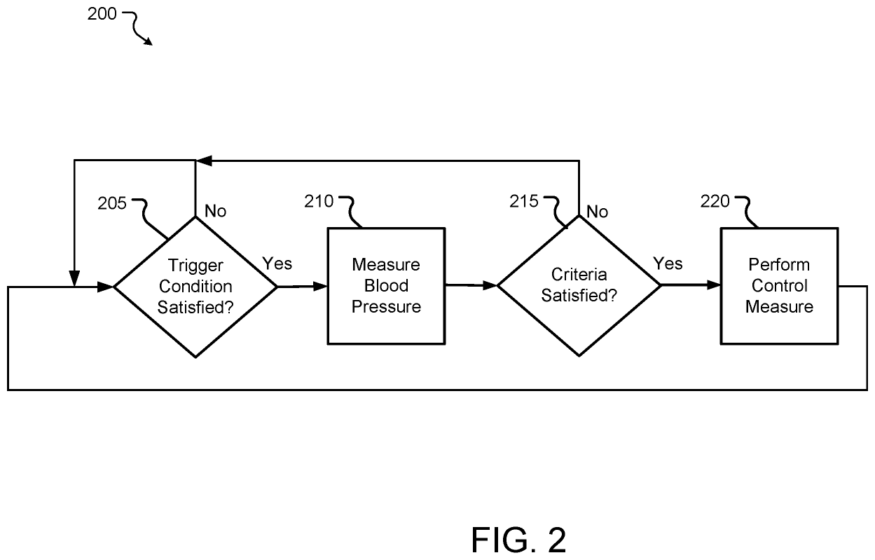

FIG. 2 illustrates an example of a process flow 200 in accordance with aspects of the present disclosure. In the following description of the process flow 200, the operations may be performed in a different order than the order shown, or the operations may be performed in different orders or at different times. Certain operations may also be left out of the process flow 200, or other operations may be added to the process flow 200.

It is to be understood that while a computing device 102, a sensor device 112, and a stimulation device 122 are described as performing a number of the operations of process flow 200, any device (e.g., another computing device 102, sensor device 112, and/or stimulation device 122) may perform the operations shown.

The process flow 200 (and/or one or more operations thereof) may be carried out or otherwise performed, for example, by at least one processor. The at least one processor may be the same as or similar to the processor(s) 104 of the computing device 102 described above. A processor other than any processor described herein may also be used to execute the process flow 200. The at least one processor may perform operations of the process flow 200 by executing elements stored in a memory such as the memory 106. The elements stored in memory and executed by the processor may cause the processor to execute one or more operations of a function as shown in the process flow 200. One or more portions of the process flow 200 may be performed by the processor executing any of the contents of memory (e.g., monitoring engine 126, machine learning model(s) 138, processing algorithm(s) 142, etc.). Aspects of the process flow are described with reference to FIG. 1B.

At 205, the computing device 102 may determine whether a trigger condition is satisfied. The trigger condition may include any example trigger condition (or combination of trigger conditions) as described herein. For example, the trigger condition may include temporal criteria (e.g., expiry of a temporal value, for example, 15 minutes), a change in posture of the subject 148 (e.g., from a sitting position to a standing position, from a position lying down to a sitting position, etc.), a change in value of a biometric parameter 152 (e.g., a change in heart rate), a change in activity level associated with the subject 148 (e.g., from a relatively low activity level to a relatively high activity level), and/or a control signal associated with delivering a therapy treatment (e.g., pump bolus).

At 210, the computing device 102 may measure (or derive) a blood pressure value of the subject 148 based on the comparison result of 205 (e.g., “Trigger condition satisfied”=Yes). The computing device 102 may measure (or derive) the blood pressure value, for example, using any of the example techniques described herein. Additionally, or alternatively, the computing device 102 may refrain from measuring (or deriving) a blood pressure value based on the comparison result of 205 (e.g., “Trigger condition satisfied”=No).

At 215, the computing device 102 may determine whether the blood pressure value satisfies one or more criteria. For example, the computing device 102 may compare the blood pressure value to a target blood pressure range (e.g., a range corresponding to high blood pressure, a range corresponding to low blood pressure, etc.).

At 220, the computing device 102 may perform a control measure described herein based on the comparison of 215 (e.g., “Criteria Satisfied”=Yes). For example, the computing device 102 may deliver therapy treatment to the subject 148 via the stimulation device 122 and/or output a notification (e.g., via user interface 110) indicating an action associated with treating a medical condition. In some aspects, the computing device 102 may adjust one or more parameters associated with delivering therapy treatment to the subject 148. Additionally, or alternatively, the computing device 102 may refrain from performing a control measure based on the comparison at 215 (e.g., “Criteria Satisfied”=No).

As described herein, control measures supported by the present disclosure may include pressure or relative pressure. Control measures may include fiducial points or timing intervals on pulse pressure waveform (patient-dependent). The control measures may include sensing of baroreceptor firing (e.g., frequency and patterns), which may confirm drug delivery.

The computing device 102 may iteratively perform any combination of the operations described with reference to 205 through 220, thereby supporting closed loop blood pressure control.

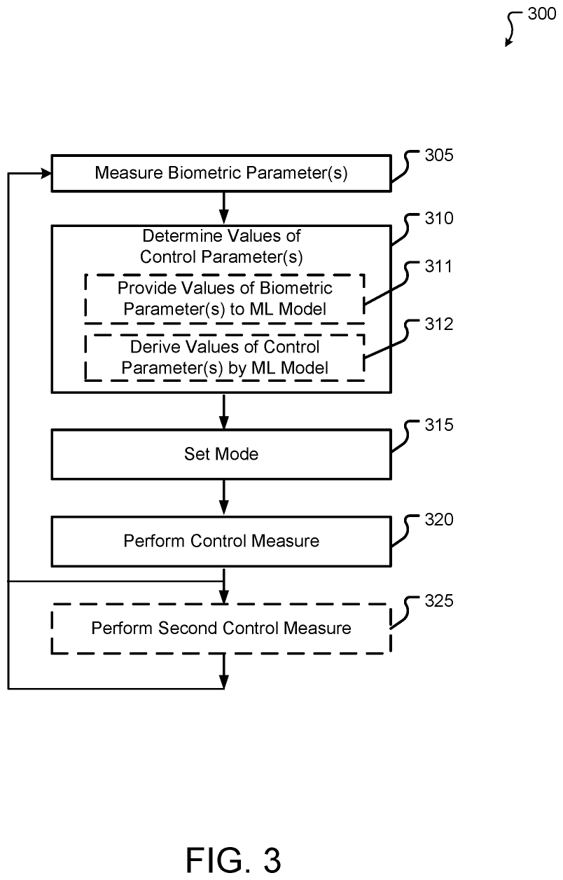

FIG. 3 illustrates an example of a process flow 300 in accordance with aspects of the present disclosure. In some examples, process flow 300 may implement aspects of the system 100 described herein.

In the following description of the process flow 300, the operations may be performed in a different order than the order shown, or the operations may be performed in different orders or at different times. Certain operations may also be left out of the process flow 300, or other operations may be added to the process flow 300.

It is to be understood that any of the operations of process flow 300 may be performed by any device (e.g., a computing device 102, another computing device 102, a server, etc.).

The process flow 300 (and/or one or more operations thereof) may be carried out or otherwise performed, for example, by at least one processor. The at least one processor may be the same as or similar to the processor(s) 104 of the computing device 102 described above. A processor other than any processor described herein may also be used to execute the process flow 300. The at least one processor may perform operations of the process flow 300 by executing elements stored in a memory such as the memory 106. The elements stored in memory and executed by the processor may cause the processor to execute one or more operations of a function as shown in the process flow 300. One or more portions of the process flow 300 may be performed by the processor executing any of the contents of memory (e.g., monitoring engine 126, machine learning model(s) 138, processing algorithm(s) 142, etc.).

At 305, the process flow 300 includes measuring, by one or more sensors (e.g., one or more sensor devices 112 of FIG. 1), at least one biometric parameter associated with a subject.

At 310, the process flow 300 includes determining values of one or more control parameters based on measuring the at least one biometric parameter.

In some aspects, the one or more sensors may include an optical sensor, an ultrasound sensor, or both. In an example, the at least one biometric parameter may include a diameter associated with an anatomical element (e.g., an artery, vein, etc.) of the subject, a flow through the anatomical element, or both. In another example, the at least one biometric parameter may include a pulse wave velocity value associated with the anatomical element.

In some aspects, the one or more sensors may include an optical sensor. In an example, the at least one biometric parameter may include a characteristic of a pulse (e.g., generated based on measurements by the one or more sensors) associated with an anatomical element (e.g., an artery, vein, etc.) of the subject.

In some examples, the characteristic may be a shape of the pulse. In some other examples, the characteristic may be a delta value between a maximum value (e.g., peak value) of the pulse and a minimum value of the pulse. In some other examples, the characteristic may be a ratio between the maximum value and the minimum value of the pulse. In some embodiments, the characteristic may be identified as fiducial points, area under the curve, timing intervals, 1^{st}, 2^{nd}, and 3^{rd}, derivatives, or the like associated with a blood pressure waveform provided by the optical sensor. In some cases, the characteristic may be a waveform shape, and measuring the at least one biometric parameter may include comparing the blood pressure waveform to a waveform template (e.g., waveform template matching).

In some aspects, the one or more sensors may include an impedance sensor. In an example, the at least one biometric parameter may include a characteristic of a pulse of an impedance measurement (e.g., provided by the impedance sensor) associated with an anatomical element of the subject.

In some examples, the characteristic may be a pulse shape of the impedance measurement. In some other examples, the characteristic may be a delta value between a maximum value (e.g., peak value) of the pulse of the impedance measurement and a minimum value of the pulse. In some other examples, the characteristic may be a ratio between the maximum value and the minimum value of the pulse. In some embodiments, the characteristic may be identified as fiducial points, area under the curve, timing intervals, 1^{st}, 2^{nd}, and 3^{rd}, derivatives, or the like associated with a waveform provided by the impedance sensor. In some cases, the characteristic may be a waveform shape, and measuring the at least one biometric parameter may include comparing the waveform indicative of impedance to a waveform template (e.g., waveform template matching).

In some aspects, the one or more sensors may include a subcutaneous blood pressure sensor, and the at least one biometric parameter may include blood pressure (e.g., measured by the subcutaneous blood pressure sensor) associated with the subject. In an example, the one or more control parameters may include a characteristic associated with the blood pressure.

In some aspects, the at least one biometric parameter may include: a pulse shape, a heart rate, blood volume, a flow rate, a pressure, or a combination thereof in association with an anatomical element of the subject. In some aspects, the at least one biometric parameter may include: a first marker of a kidney function associated with blood pressure (e.g., high blood pressure, measured blood pressure that exceeds a threshold value) of the subject, a second marker of a thyroid function associated with the blood pressure, or a combination thereof.

In some example aspects, determining the values of the one or more control parameters may include deriving (e.g., using an algorithm described herein, for example, a machine learning algorithm) the values of the one or more control parameters based on measuring the at least one biometric parameter. In an example, the one or more control parameters may include blood pressure of the subject. For example, the process flow 300 may include providing (at 311) a set of values of the at least one biometric parameter to a machine learning model (e.g., machine learning model 138 described with reference to FIG. 1). Determining the values of the one or more control parameters may include deriving (at 312), by the machine learning model, the values of the one or more control parameters in response to the machine learning model processing the set of values of the at least one biometric parameter.

At 315, the process flow 300 includes setting a mode associated with performing a control measure. In some aspects, setting the mode is based on a value of the at least one biometric parameter, a value of the one or more control parameters, or both.

Non-limiting examples of the control measure include, controlling pulse width, enabling a continuous mode or burst mode (e.g., bursts of pulses or signals), on-off cycling of the burst, intensity of an electrical current administered to the subject, selection of an electrode associated with administering treatment (e.g., electrical stimulation) to the subject, selection of a lead associated with administering the treatment, selection of a location (e.g., anatomical element, position on the subject, etc.) associated with administering the treatment, selection of a cathode-anode associated with administering the treatment, and selecting a pulse form of an electrical signal associated with administering the treatment.

At 320, the process flow 300 includes performing the control measure based on a comparison of the values of the one or more control parameters to a threshold.

In some aspects, the at least one biometric parameter may include an optical signal associated with an anatomical element, an electrical impedance associated with the anatomical element, or a pressure associated with the anatomical element. In an example, determining the values of the one or more control parameters (at 310) may include deriving a waveform representation of the optical signal, the electrical impedance, or the pressure. In some aspects, performing the control measure (at 320) is based on comparing one or more characteristics of the waveform representation to a set of threshold criteria.

In some examples, the pressure may be intra-arterial blood pressure associated with the anatomical element, and the intra-arterial blood pressure may be measured by an optical sensor or impedance sensor described herein. In some other examples, the pressure may be a transmural pressure associated with the anatomical element. The transmural pressure may be, for example, a significantly attenuated in comparison to intra-arterial blood pressure. The transmural pressure, for example, may indicate a change in relative intra-arterial blood pressure.

Additionally, or alternatively, at 325, the process flow 300 includes performing a second control measure based on a set of data points (e.g., fiducial points, or a subset of fiducial points as described herein) associated with the at least one biometric parameter.

In some aspects, at 325, the process flow 300 may include using the data points as a control signal for performing the second control measure. For example, performing the second control measure may include refraining from converting the data points to values of a control parameter (e.g., blood pressure).

In some aspects, performing the control measure at 320 (and additionally, or alternatively, performing the second control measure at 325) may include outputting a control signal to a device. In an example, performing the control measure may include delivering, by the device, therapy treatment to the subject based on the control signal. In some examples, performing the control measure may include outputting, by the device, a notification based on the control signal. In some aspects, the notification may include an indication of one or more actions (e.g., take prescribed medication, perform a prescribed action (for example, “walk for 10 minutes”), etc.) associated with treating a medical condition.

In some aspects, performing the control measure at 320 (and additionally, or alternatively, performing the second control measure at 325) may include selecting a mode of a device configured for delivering therapy treatment to the subject and delivering the therapy treatment to the subject based on the mode. In an example, the device may include a pacemaker, a neurostimulator, a drug pump, or a mechanical device.

In some aspects, measuring the at least one biometric parameter (at 305), determining the values of the one or more control parameters (at 310), and/or performing the control measure (at 320) is in response to one or more trigger criteria.

In some aspects, the one or more trigger criteria may include temporal criteria. In some aspects, the one or more trigger criteria may include a change in posture of the subject (e.g., from a sitting position to a standing position, from a position lying down to a sitting position, etc.). In some aspects, the one or more trigger criteria may include a change in value of the at least one biometric parameter (e.g., a change in heart rate). In some aspects, the one or more trigger criteria may include a change in activity level (e.g., from a relatively low activity level to a relatively high activity level) or activity status associated with the subject.

In some aspects, the one or more trigger criteria may include a control signal associated with delivering therapy treatment to the subject. In some other aspects, the one or more trigger criteria may include delivery of the therapy treatment by the device.

In some aspects, the one or more trigger criteria comprise two or more trigger criteria. In an example, the process flow 300 may include deriving a composite trigger value based on the two or more trigger criteria and respective weights of the two or more trigger criteria. In some aspects, performing the control measure (at 320) is in response to the composite parameter value satisfying one or more threshold values. For example, performing the control measure (at 320) may be in response to the composite parameter value exceeding a minimum threshold value and a being less than maximum threshold value in association with preventing overstimulation. That is, for example, performing the control measure (at 320) may be in response to the composite parameter value being included in a threshold range or a window.

In some aspects, measuring the at least one biometric parameter, determining the values of the one or more control parameters, and performing the control measure are associated with a closed loop operating mode of the system.

As noted above, the present disclosure encompasses methods with fewer than all of the steps identified in FIG. 3 (and the corresponding description of the process flow 300), as well as methods that include additional steps beyond those identified in FIG. 3 (and the corresponding description of the process flow 300). The present disclosure also encompasses methods that comprise one or more steps from one method described herein, and one or more steps from another method described herein.

The foregoing is not intended to limit the disclosure to the form or forms disclosed herein. In the foregoing Detailed Description, for example, various features of the disclosure are grouped together in one or more aspects, implementations, and/or configurations for the purpose of streamlining the disclosure. The features of the aspects, implementations, and/or configurations of the disclosure may be combined in alternate aspects, implementations, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, implementation, and/or configuration. Thus, the following claims are hereby incorporated into this Detailed Description, with each claim standing on its own as a separate preferred implementation of the disclosure.

Moreover, though the foregoing has included description of one or more aspects, implementations, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, implementations, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

Example Aspects of the Present Disclosure Include:

A system including: a processor; and a memory storing instructions thereon that, when executed by the processor, cause the processor to: measure, by one or more sensors, at least one biometric parameter associated with a subject; determine values of one or more control parameters based on measuring the at least one biometric parameter; and perform a control measure based on a comparison of the values of the one or more control parameters to a threshold.

Any of the aspects herein, wherein: determining the values of the one or more control parameters includes deriving the values of the one or more control parameters based on measuring the at least one biometric parameter; and the one or more control parameters include blood pressure of the subject.

Any of the aspects herein, wherein the instructions are further executable by the processor to: perform a second control measure based on a set of data points associated with the at least one biometric parameter.

Any of the aspects herein, wherein: the one or more sensors include an optical sensor, an ultrasound sensor, or both; and the at least one biometric parameter includes: a diameter associated with an anatomical element of the subject, a flow through the anatomical element, or both; or a pulse wave velocity value associated with the anatomical element.

Any of the aspects herein, wherein: the one or more sensors include an optical sensor; and the at least one biometric parameter includes a characteristic of a pulse associated with an anatomical element of the subject.

Any of the aspects herein, wherein: the one or more sensors include an impedance sensor; and the at least one biometric parameter includes a characteristic of a pulse of an impedance measurement associated with an anatomical element of the subject.

Any of the aspects herein, wherein: the one or more sensors include a subcutaneous blood pressure sensor; the at least one biometric parameter includes blood pressure associated with the subject; and the one or more control parameters include a characteristic associated with the blood pressure.

Any of the aspects herein, wherein the at least one biometric parameter includes: a pulse shape, a heart rate, blood volume, a flow rate, a pressure, or a combination thereof in association with an anatomical element of the subject.

Any of the aspects herein, wherein: the at least one biometric parameter includes an optical signal associated with an anatomical element, an electrical impedance associated with the anatomical element, or a pressure associated with the anatomical element; determining the values of the one or more control parameters includes deriving a waveform representation of the optical signal, the electrical impedance, or the pressure; and performing the control measure is based on comparing one or more characteristics of the waveform representation to a set of threshold criteria.

Any of the aspects herein, wherein performing the control measure includes: outputting a control signal to a device; and at least one of: delivering, by the device, therapy treatment to the subject based on the control signal; and outputting, by the device, a notification based on the control signal, wherein the notification includes an indication of one or more actions associated with treating a medical condition.

Any of the aspects herein, wherein performing the control measure includes: selecting a mode of a device configured for delivering therapy treatment to the subject, the device including a pacemaker, a neurostimulator, a drug pump, or a mechanical device; and delivering the therapy treatment to the subject based on the mode.

Any of the aspects herein, wherein the instructions are further executable by the processor to: set a mode associated with performing the control measure, wherein setting the mode is based on a value of the at least one biometric parameter, a value of the one or more control parameters, or both.

Any of the aspects herein, wherein measuring the at least one biometric parameter, determining the values of the one or more control parameters, and performing the control measure is in response to one or more trigger criteria.

Any of the aspects herein, wherein the one or more trigger criteria include at least one of: temporal criteria; a change in posture of the subject; a change in value of the at least one biometric parameter; and a change in activity level or activity status associated with the subject.

Any of the aspects herein, wherein the one or more trigger criteria include at least one of: a control signal associated with delivering therapy treatment to the subject; and delivery of the therapy treatment by a device.

Any of the aspects herein, wherein: the one or more trigger criteria include two or more trigger criteria; and the instructions are further executable by the processor to: derive a composite trigger value based on the two or more trigger criteria and respective weights of the two or more trigger criteria, wherein performing the control measure is in response to the composite parameter value satisfying one or more threshold values.

Any of the aspects herein, further including: providing a set of values of the at least one biometric parameter to a machine learning model, wherein determining the values of the one or more control parameters includes deriving, by the machine learning model, the values of the one or more control parameters in response to the machine learning model processing the set of values of the at least one biometric parameter.

Any of the aspects herein, wherein measuring the at least one biometric parameter, determining the values of the one or more control parameters, and performing the control measure are associated with a closed loop operating mode of the system.

A method including: measuring, by one or more sensors, at least one biometric parameter associated with a subject; determining values of one or more control parameters based on measuring the at least one biometric parameter; and performing a control measure based on a comparison of the values of the one or more control parameters to a threshold.

A system including: one or more sensors; a device configured for delivering therapy treatment; a processor; and a memory storing instructions thereon that, when executed by the processor, cause the processor to: measure, by the one or more sensors, at least one biometric parameter associated with a subject; determine values of one or more control parameters based on measuring the at least one biometric parameter; and perform a control measure via the device based on a comparison of the values of the one or more control parameters to a threshold.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an implementation that is entirely hardware, an implementation that is entirely software (including firmware, resident software, micro-code, etc.) or an implementation combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electromagnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A system comprising:
a processor; and
a memory storing instructions thereon that, when executed by the processor, cause the processor to:
measure, by one or more sensors, at least one biometric parameter associated with a subject;
determine values of one or more control parameters based on measuring the at least one biometric parameter; and
perform a control measure based on a comparison of the values of the one or more control parameters to a threshold.

2. The system of claim 1, wherein:
determining the values of the one or more control parameters comprises deriving the values of the one or more control parameters based on measuring the at least one biometric parameter; and
the one or more control parameters comprise blood pressure of the subject.

3. The system of claim 1, wherein the instructions are further executable by the processor to:
perform a second control measure based on a set of data points associated with the at least one biometric parameter.

4. The system of claim 1, wherein:
the one or more sensors comprise an optical sensor, an ultrasound sensor, or both; and
the at least one biometric parameter comprises:
a diameter associated with an anatomical element of the subject, a flow through the anatomical element, or both; or
a pulse wave velocity value associated with the anatomical element.

5. The system of claim 1, wherein:
the one or more sensors comprise an optical sensor; and
the at least one biometric parameter comprises a characteristic of a pulse associated with an anatomical element of the subject.

6. The system of claim 1, wherein:
the one or more sensors comprise an impedance sensor; and
the at least one biometric parameter comprises a characteristic of a pulse of an impedance measurement associated with an anatomical element of the subject.

7. The system of claim 1, wherein:
the one or more sensors comprise a subcutaneous blood pressure sensor;
the at least one biometric parameter comprises blood pressure associated with the subject; and
the one or more control parameters comprise a characteristic associated with the blood pressure.

8. The system of claim 1, wherein the at least one biometric parameter comprises at least one of:
a pulse shape, a heart rate, blood volume, a flow rate, a pressure, or a combination thereof in association with an anatomical element of the subject; and
a first marker of a kidney function associated with blood pressure of the subject, a second marker of a thyroid function associated with the blood pressure, or a combination thereof.

9. The system of claim 1, wherein:
the at least one biometric parameter comprises an optical signal associated with an anatomical element, an electrical impedance associated with the anatomical element, or a pressure associated with the anatomical element;
determining the values of the one or more control parameters comprises deriving a waveform representation of the optical signal, the electrical impedance, or the pressure; and
performing the control measure is based on comparing one or more characteristics of the waveform representation to a set of threshold criteria.

10. The system of claim 1, wherein performing the control measure comprises:
outputting a control signal to a device; and
at least one of:
delivering, by the device, therapy treatment to the subject based on the control signal; and
outputting, by the device, a notification based on the control signal, wherein the notification comprises an indication of one or more actions associated with treating a medical condition.

11. The system of claim 1, wherein performing the control measure comprises:
selecting a mode of a device configured for delivering therapy treatment to the subject, the device comprising a pacemaker, a neurostimulator, a drug pump, or a mechanical device; and
delivering the therapy treatment to the subject based on the mode.

12. The system of claim 1, wherein the instructions are further executable by the processor to:
set a mode associated with performing the control measure,
wherein setting the mode is based on a value of the at least one biometric parameter, a value of the one or more control parameters, or both.

13. The system of claim 1, wherein measuring the at least one biometric parameter, determining the values of the one or more control parameters, and performing the control measure is in response to one or more trigger criteria.

14. The system of claim 13, wherein the one or more trigger criteria comprise at least one of:
temporal criteria;
a change in posture of the subject;
a change in value of the at least one biometric parameter; and
a change in activity level or activity status associated with the subject.

15. The system of claim 13, wherein the one or more trigger criteria comprise at least one of:
a control signal associated with delivering therapy treatment to the subject; and
delivery of the therapy treatment by a device.

16. The system of claim 13, wherein:
the one or more trigger criteria comprise two or more trigger criteria; and
the instructions are further executable by the processor to:
derive a composite trigger value based on the two or more trigger criteria and respective weights of the two or more trigger criteria,
wherein performing the control measure is in response to the composite parameter value satisfying one or more threshold values.

17. The system of claim 1, further comprising:
providing a set of values of the at least one biometric parameter to a machine learning model,
wherein determining the values of the one or more control parameters comprises deriving, by the machine learning model, the values of the one or more control parameters in response to the machine learning model processing the set of values of the at least one biometric parameter.

18. The system of claim 1, wherein measuring the at least one biometric parameter, determining the values of the one or more control parameters, and performing the control measure are associated with a closed loop operating mode of the system.

19. A method comprising:
measuring, by one or more sensors, at least one biometric parameter associated with a subject;
determining values of one or more control parameters based on measuring the at least one biometric parameter; and
performing a control measure based on a comparison of the values of the one or more control parameters to a threshold.

20. A system comprising:
one or more sensors;
a device configured for delivering therapy treatment;
a processor; and
a memory storing instructions thereon that, when executed by the processor, cause the processor to:
measure, by the one or more sensors, at least one biometric parameter associated with a subject;
determine values of one or more control parameters based on measuring the at least one biometric parameter; and
perform a control measure via the device based on a comparison of the values of the one or more control parameters to a threshold.
